(19) United States
(12) Patent Application Publication
(10) Pub. No.: US 2022/0313969 A1
(43) Pub. Date: Oct. 6, 2022

(54) METHODS FOR TREATMENT OF THE SKIN

(71) Applicant: NATALIA BOLEYN, LOS ANGELES, CA (US)

(72) Inventor: NATALIA BOLEYN, LOS ANGELES, CA (US)

(21) Appl. No.: 17/219,415

(22) Filed: Mar. 31, 2021

(51) Int. Cl. A61M 37/00; A61Q 19/00; A61K 8/92

(52) U.S. Cl. CPC A61M 37/0076; A61Q 19/00; A61K 8/922; A61M 2202/08; A61M 2202/0007; A61M 2210/04

(57) ABSTRACT

A method of treating the skin is disclosed that comprises placing a composition including frankincense essential oil on one or more needles; piercing the skin within the boundary of a stretch mark with the one or more needles to a depth between 0.5 mm and 2 mm; and repeating the piercing step over the area of the stretch mark.

### FIELD

The present patent document generally relates to methods and processes for treatment of the skin. In particular, the present patent document relates to the treatment of striae distensae lesions, also known as stretch marks.

### BACKGROUND

Striae distensae lesions, also known as stretch marks, form for many different reasons. Different individuals have different propensities for getting stretch marks. Regardless, most people do not like their appearance and wish to have their appearance reduced or completely removed.

Numerous topical creams and ointments have been proposed. U.S. Patent Publication No. 2018/0020075 describes a topical essential oil composition for the treatment of stretch marks. U.S. Pat. No. 8,765,198 (“'198 Patent”) describes a treatment that may be administered topically or orally to treat stretch marks. The background section of the '198 Patent further describes a history of many other patents that discuss various treatments for stretch marks.

Clearly, the treatment of stretch marks is of value to the public and a better method would help improve the ability of people to treat or have their stretch marks treated.

### SUMMARY OF THE EMBODIMENTS

Objects of the present patent document are to provide improved methods of treating the skin and in particular improved methods of treating stretch marks. In preferred embodiments a method of treating the skin is provided. In preferred embodiments, the method comprises: placing a composition including frankincense essential oil on one or more needles; piercing the skin within a boundary of a stretch mark with the one or more needles to a depth between 0.5 mm and 2 mm; and repeating the piercing step over the area of the stretch mark.

As used herein, the term “within the boundary of” means generally within the area that is considered the stretch mark but can include punctures outside this area. To be clear, the scope is meant to be inclusive of also making punctures outside the boundary area as long as punctures are made within the boundary area.

In preferred embodiments, a tattoo gun is used to pierce the skin with the one or more needles. In some embodiments, the area of the stretch mark is punctured between 20 times per square inch and 1500 times per square inch. In other embodiments, the area of the stretch mark is punctured between 10 times per square inch and 100 times per square inch. In yet other embodiments, the area of the stretch mark is punctured between 20 times per square inch and 1500 times per square inch. In still yet other embodiments, the area of the stretch mark is punctured between 50 times per square inch and 1000 times per square inch.

In some embodiments, the one or more needles has a diameter between 0.30 mm and 0.35 mm. In yet other embodiments, other diameters may be used. Ideally, a magnum tattoo needle is used. In preferred embodiments, the magnum tattoo needle is two rows of needles in a weave formation.

In preferred embodiments, the skin is pierced between 50 and 3000 times per minute.

Ideally, the composition that is injected into the affected area has special properties to promote healing. In preferred embodiments, the composition further comprises avocado oil and sunflower oil. In still yet more preferred embodiments, the composition further comprises water, glycerine, Hamamelis virginiana extract, cetylpyridinium chloride and cocamidopropyl betaine.

In some embodiments, the composition includes between 0.1% and 5% frankincense essential oil.

### BRIEF DESCRIPTION OF THE DRAWINGS

FIG. 1 illustrates a first needle pattern for use in treating the skin with a composition.

FIG. 2 illustrates a second needle pattern for use in treating the skin with a composition.

### DETAILED DESCRIPTION OF THE PREFERRED EMBODIMENTS

Unlike previous methods of treatment which apply a composition topically or even take the composition orally, the methods herein reduce or minimize the appearance of stretch marks by injecting a composition into the stretch mark.

Preferably, a needle is used to pierce the skin in the location of the stretch mark to a depth between 0.1 mm and 5 mm and more preferably to a depth between 0.5 mm and 2 mm. In preferred embodiments, the needle is used to inject the composition into the dermis layer of the skin below the stretch mark. The composition is injected into the skin by applying it to the skin and/or the needle and then repeatedly piercing the skin with the needle. An area of the skin may be pierced repeatedly in order to work the composition into the skin. In preferred embodiments, a tattoo gun may be used to accomplish the skin piercing with the needle.

In preferred embodiments, between 20 punctures per square inch and 3000 punctures per square inch are used to treat the stretch mark area. In even more preferred embodiments, between 50 punctures per square inch and 1000 punctures per square inch is used.

In preferred embodiments, the entire area of the stretch mark may be covered with punctures with minimal spacing between punctures. Minimal spacing means that on average only 0.1 mm to 1 mm spacing is left between punctures.

Typical tattoo guns can puncture the skin between 50 and 3000 times per minute and in preferred embodiments, the composition is injected into the skin where the stretch mark appears at a rate of 50 to 3000 times per minute. In a more preferred embodiment, the composition is injected into the skin at a rate of 1375 to 2100 times per minute. In an even more preferred embodiment, the composition is injected into the skin at a rate of 1600 to 2000 times per minute.

In preferred embodiments, the needle is a solid needle as is typical in tattoo needles and not a hollow needle like used in a syringe. However, any shape needle may be used. If a tattoo gun is used, the needle will need to be compatible to the gun. The needle may range between a diameter of 0.15 mm to 0.40 mm. More preferably, the needle has a diameter of between 0.30 mm to 0.35 mm.

There are different style tattoo needles and any of them may be used but preferably a magnum tattoo needle is used. Magnum tattoo needles that may be used include round/curved.

In preferred embodiments, a single magnum needle is used that is comprised by more than one physical needle. Anywhere from 1 to 50 physical needles may be used at once as part of a single Magnum needle. Preferably, between 3 and 13 needles are used and more preferably between 5 and 9 needles are used. When more than one needle is used, needles may be grouped in many different ways. In preferred embodiments, needles are grouped in two rows, one on top of the other in a weaved formation.

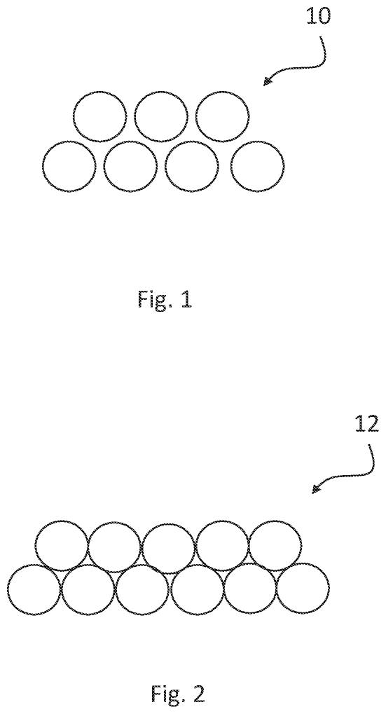

FIG. 1 illustrates a first needle pattern 10 for use in treating the skin with a composition. As you can see in FIG. 1, two rows of needles are used. The rows are parallel and the needles are spaced a small distance apart. There are three needles in the first row and four needles in the second row. FIG. 2 illustrates a second needle pattern 12 for use in treating the skin with a composition. As you can see in FIG. 2, two rows of needles are used. The rows are parallel and the needles are spaced as close together as possible. There are five needles in the first row and six needles in the second row. As may be appreciated, in other embodiments the rows could have different numbers of needles and the spacing or pattern could be different.

In preferred embodiments that use a tattoo gun, the tattoo gun can be set to a speed ranging between 1v and 18v and more preferably to a speed ranging between 5.5v and 8.5v.

In preferred embodiments, an area containing stretch marks is cleaned and a tattoo gun is loaded with the appropriate needle and set to the appropriate speed and depth. The composition is applied to the needle and/or the surface of the skin to be treated. The area of the stretch mark is then punctured repeatedly with the needle injecting the composition into the epidermis and dermis below in the area of the stretch mark. To this end, it could be said that the stretch mark is literally tattooed with the composition.

In some embodiments, clients may notice an improvement between 20% and 100% after a single treatment. In other embodiments, treatments may be repeated and the same area may be treated by injecting a composition with a tattoo device over a period of repeated treatments. In preferred embodiments, the same area may be treated between 2 and 10 times.

In different embodiments, the composition that is injected into the tattoo could be any type of composition. Preferably, the composition is oil based. Examples of oils include, but are not limited to, sweet almond oil, olive oil, wheat germ oil, castor oil, avocado oil, rosehip seed oil, calendula oil, jojoba oil, grape seed oil, apricot kernel oil, flaxseed oil, hazelnut oil, walnut oil, pecan nut oil, macadamia nut oil, and vegetable oils such as for example safflower oil, sunflower oil, canola oil, soybean oil, and peanut oil, frankincense essential oil, bergamot oil, clary sage oil, lavender oil, juniper oil, pine oil, marjoram oil, sandalwood oil, rosemary oil, citrus oils and combinations thereof.

The composition may also include vitamins such as (but not limited) vitamin A, vitamin Bs such as vitamin B3, vitamin B5, and vitamin B12, vitamin C, vitamin K, and vitamin E. The composition may also include essential oils. Examples of essential oils include, but are not limited to: bergamot, clary sage, lavender, juniper, pine, marjoram, sandalwood, rosemary, frankincense, and citrus oils and derivatives thereof.

The composition may also contain conventional cosmetic additives, such as for example colorants and fragrances.

The compositions may contain preservatives, humectants, anti-microbial agents and the like to stabilize the composition and prolong its shelf life

In preferred embodiments, the composition includes frankincense essential oil. Other ingredients mentioned above may be combined with the frankincense essential oil. In one embodiment, the composition comprises frankincense essential oil, avocado oil and sunflower oil. In yet other embodiments, the composition comprises frankincense essential oil, avocado oil and sunflower oil mixed with other ingredients.

In some embodiments, the composition may further include water, glycerine, Hamamelis virginiana extract, cetylpyridinium chloride and cocamidopropyl betaine. Any of these ingredients may be used in combination with any other ingredients.

In a preferred embodiment, the composition may consist of avocado oil, sunflower oil, water, glycerin, Hamamelis virginiana extract, cetylpyridinium chloride, cocamidopropyl betaine and frankincense essential oil. Main ingredient is frankincense essential oil. In other embodiments, it may be any combination of fewer of these components.

In preferred embodiments, the composition includes between 20% and 50% avocado oil. In some embodiments, the composition includes between 20% and 50% sunflower oil. In some embodiments the composition contains between 10% and 40% water. In some embodiments, the composition contains between 5% and 40% glycerin. In some embodiments, the composition includes between 2% and 30% Hamamelis virginiana extract. In some embodiments, the composition includes between 1% and 25% cetylpyridinium chloride. In some embodiments, the composition includes between 1% and 25% cocamidopropyl betaine. In some embodiments, the composition includes between 0.1% and 5% frankincense essential oil.

What is claimed is:

1. A method of treating the skin comprising:
placing a composition including frankincense essential oil on one or more needles;
piercing the skin within a boundary of a stretch mark with the one or more needles to a depth between 0.5 mm and 2 mm; and
repeating the piercing step over the area of the stretch mark.

2. The method of claim 1, wherein a tattoo gun is used to pierce the skin with the one or more needles.

3. The method of claim 1, wherein the area of the stretch mark is punctured between 20 times per square inch and 100 times per square inch.

4. The method of claim 1, wherein the one or more needles has a diameter between 0.30 mm and 0.35 mm.

5. The method of claim 1, wherein the one or more needles comprise a magnum tattoo needle.

6. The method of claim 1, wherein the magnum tattoo needle is two rows of needles in a weave formation.

7. The method of claim 1, wherein the skin is pierced between 50 and 3000 times per minute.

8. The method of claim 1, wherein the composition further comprises avocado oil and sunflower oil.

9. The method of claim 8, wherein the composition further comprises water, glycerine, Hamamelis virginiana extract, cetylpyridinium chloride and cocamidopropyl betaine.

10. The method of claim 1, wherein the composition includes between 0.1% and 3% frankincense essential oil.

11. A method of treating the skin comprising:
placing a composition including frankincense essential oil on one or more tattoo needles; and
using a tattoo gun to repeatedly pierce the skin within a boundary of a stretch mark with the one or more needles to a depth between 0.5 mm and 2 mm.

12. The method of claim 11, wherein the area of the stretch mark is punctured between 10 times per square inch and 100 times per square inch.

13. The method of claim 11, wherein the one or more needles has a diameter between 0.3 mm and 0.35 mm.

14. The method of claim 11, wherein the one or more needles is a magnum tattoo needle.

15. The method of claim 11, wherein the magnum tattoo needle is two rows of needles in a weave formation.

16. The method of claim 11, wherein the skin is pierced between 50 and 3000 times per minute.

17. The method of claim 11, wherein the composition further comprises avocado oil and sunflower oil.

18. The method of claim 17, wherein the composition further comprises water, glycerine, Hamamelis virginiana extract, cetylpyridinium chloride and cocamidopropyl betaine.

19. A method of treating the skin comprising:
placing a composition including frankincense essential oil, avocado oil, and sunflower oil on one or more tattoo needles; and
using a tattoo gun to repeatedly pierce the skin within a boundary of a stretch mark with the one or more needles to a depth between 0.5 mm and 2 mm.

20. The method of claim 19, wherein the area of the stretch mark is punctured between 10 times per square inch and 100 times per square inch
